(19) United States
(12) Patent Application Publication
(10) Pub. No.: US 2018/0110799 A1
(43) Pub. Date: Apr. 26, 2018

(54) Compositions and Methods for the Inhibition of Chondrogenesis

(71) Applicant: The Children's Hospital of Philadelphia, Philadelphia, PA (US)

(72) Inventors: Maurizio Pacifici, Philadelphia, PA (US); Julianne Huegel, Philadelphia, PA (US)

(21) Appl. No.: 15/506,107

(22) PCT Filed: Sep. 2, 2015
(86) PCT No.: PCT/US15/48013

§ 371 Date: Feb. 23, 2017

Related U.S. Application Data
(60) Provisional application No. 62/044,735, filed on Sep. 2, 2014.

(51) Int. Cl. A61K 31/727; A61P 19/00

(52) U.S. Cl. CPC A61K 31/727; A61P 19/00

(57) ABSTRACT

Compositions and methods for inhibiting chondrogenesis are disclosed.

This application claims priority under 35 U.S.C. § 119(e) to U.S. Provisional Patent Application No. 62/044,735, filed Sep. 2, 2014. The foregoing application is incorporated by reference herein.

This invention was made with government support under Grant Number R01 AR061758 awarded by the National Institutes of Health. The government has certain rights in the invention.

### FIELD OF THE INVENTION

The present invention relates to the fields of chondrogenesis. More specifically, the invention provides compositions and methods for inhibiting chondrogenesis and the treatment of related diseases.

### BACKGROUND OF THE INVENTION

Several publications and patent documents are cited throughout the specification in order to describe the state of the art to which this invention pertains. Each of these citations is incorporated herein by reference as though set forth in full.

Heparanase is a multifunctional protein that is involved in a variety of physiological and pathological processes and represents the only entity of its kind encoded in the mammalian genome (Fux et al. (2009) Trends Biochem. Sci., 34:511-9; Vreys et al. (2007) J. Cell. Mol. Med., 11:427-52). The enzyme can cleave heparan sulfate (HS) chains present in syndecans, glypicans and other HS-rich proteoglycans and in so doing, affects proteoglycan homeostasis, function, mobility and internalization and can influence various processes including cell spreading, migration and proliferation (Freeman et al. (1998) Biochem J., 330:1341-50; Levy-Adam et al. (2008) PLoS ONE, 3:e2319). Importantly, cleavage of HS chains can also release HS-bound cytokines, growth factors and signaling proteins, thus enhancing their bio-availability, range of action and effects on target cells (Patel et al. (2007) Development 134:4177-86; Ramani et al. (2011) J. Biol. Chem., 286:6490-9). Latent heparanase on the cell surface can interact with syndecans, and this interaction leads to rapid internalization of the complex and delivery to lysosomes where the enzyme is activated by cathepsin L (Abbouud-Jarrous et al. (2008) J. Biol. Chem., 283:18167-76). Heparanase has additional important functions. It can lead to syndecan clustering and activation of downstream effector pathways involving PKC, Src and Racl (Levy-Adam et al. (2008) PLoS ONE, 3:e2319) and can activate β1-integrin (Zetser et al. (2003) Cancer Res., 63:7733-41), mechanisms all facilitating cell spreading and migration (Woods, A. (2001) J. Clin. Invest., 107:935-41). Because some of these actions do not appear to require enzymatic activity and can be elicited by inactive heparanase as well, they are likely to involve and rely on the non-enzymatic domains of the protein (Goldshmidt et al. (2003) FASEB J., 17:1015-25). As a reflection of its multiple and potent biological activities, heparanase is often up-regulated in human cancers and is closely associated with, and may lead to, neoplastic cell behavior and metastasis (Vreys et al. (2007) J. Cell. Mol. Med., 11:427-52; Zetser et al. (2003) Cancer Res., 63:7733-41; Hulett et al. (1999) Nat Med., 5:183-7). The protein is believed to facilitate the invasive behavior of cancer cells and tumor growth by release of extracellular matrix-bound angiogenic factors including vascular endothelial growth factor (VEGF) and by up-regulating expression of important genes such as HGF, MMP-9 and VEGF itself (Fux et al. (2009) Trends Biochem. Sci., 34:511-9; Ramani et al. (2011) J. Biol. Chem., 286:6490-9; Purushothaman et al. (2008) J. Biol. Chem., 283:32628-36). Indeed, inhibitors of heparanase administered systemically were found to reduce progression of tumor xenografts in mice (Casu et al. (2009) Pathophysiol. Haemost. Thromb., 38:195-203). These and other studies have led to the understanding that heparanase inhibition by pharmacologic strategies represents a promising and effective cancer therapy (McKenzie, E. A. (2007) Br. J. Pharmacol., 151:1-14).

Benign ectopic cartilaginous/bony tumors called exostoses characterize the pediatric skeletal disorder Hereditary Multiple Exostoses (HME) (Jones, K. B. (2011) J. Pediatr. Orthop., 31:577-86; Porter et al. (1999) J. Pathol., 188:119-25). The exostoses are growth plate-like structures that form next to, but never within, the growth plates of long bones, ribs, pelvis and other skeletal elements. Because of size and location, the exostoses can cause a variety of health problems including skeletal growth retardation and deformities, chronic pain, compression of nerves and blood vessels, and psychological concerns (Jones, K. B. (2011) J. Pediatr. Orthop., 31:577-86; Goud et al. (2012) J. Bone Joint Surg. Am., 94:1013-20; Hosalkar et al. (2007) J. Pediatr. Orthop., 27:333-7). In the majority of HME patients the exostoses remain benign through life, but in about 2 to 5% of them the exostoses progress to malignancy, turn into osteosarcomas or chondrosarcomas and thus become life-threatening (Bovee et al. (1999) Am. J. Hum. Genet., 65:689-98). Most HME patients carry heterozygous loss-of-function mutations in EXT1 or EXT2 that encode glycosyltransferases responsible for heparan sulfate (HS) synthesis (Esko et al. (2002) Annu. Rev. Biochem., 71:435-71; Jennes et al. (2009) Hum. Mutat., 30:1620-7). EXT1 and EXT2 form protein complexes in the Golgi and are both required for HS synthesis (Esko et al. (2002) Annu. Rev. Biochem., 71:435-71). HME patients thus have reduced levels—but not lack—of HS in their tissues. Puzzlingly however, the cartilaginous portions of human exostoses display barely detectable levels of HS (Hecht et al. (2005) Differentiation 73:212-21), indicating that exostosis formation may require a severe loss of HS beyond what would be caused by mere EXT haploinsufficiency. This requirement has been confirmed in transgenic mouse studies involving conditional Ext1 and/or Ext2 ablation (Jones et al. (2010) Proc. Natl. Acad. Sci., 107:2054-9; Matsumoto et al. (2010) Proc. Natl. Acad. Sci., 107:10932-7; Zak et al. (2011) 48:979-987). Studies have suggested mechanisms that could account for a severe drop of HS in human exostoses including EXT loss-of-heterozygosity, large and encompassing genomic deletions, a second hit in another gene, and background genetic traits such as modifiers (Jennes et al. (2009) Hum. Mutat., 30:1620-7; Waaijer et al. (2013) Genes Chromosomes Cancer 52:431-6; Wuyts et al. (2000) Hum. Mutat., 15:220-7). However, there are still no clear answers nor obvious genotype-phenotype correlations in HME, despite the fact that the syndrome can vary significantly in severity within affected family members and amongst individuals from different families (Pedrini et al. (2011) J. Bone Joint Surg., 93:2294-2302).

### SUMMARY OF THE INVENTION

In accordance with the instant invention, methods for inhibiting, treating, and/or preventing a chondrogenesis-related disease or disorder, such as hereditary multiple exostoses, in a subject are provided. Methods for inhibiting or preventing exostosis formation or growth in a subject are also provided. The methods of the instant invention comprise administering to a subject at least one heparanase inhibitor. In a particular embodiment, the method heparanase inhibitor is a modified heparin. The modified heparin may be glycol split, desulfated, and/or N-acetylated. In a particular embodiment, the modified heparin is roneparstat.

### BRIEF DESCRIPTION OF THE DRAWINGS

FIGS. 1A-1H show that heparanase is broadly distributed in human exostoses, but restricted in control growth plate. FIGS. 1A-1D show immunohistochemical staining of longitudinal sections of control human growth plate showing that heparanase is detectable in hypertrophic chondrocytes (hc) at the chondroosseous junction (coj) and is also prominent in perichondrium (arrowheads in FIG. 1C). Strong staining is appreciable in a blood vessel present in the secondary ossification center (FIG. 1D). FIGS. 1E-1H are sections from human exostoses stained in parallel. Heparanase staining is clear in nearly every chondrocyte regardless of location within tissue and apparent phenotypic maturation status (FIGS. 1E-1F) and also in neighboring perichondrium-like tissue (FIG. 1G, arrowheads). Clusters of large hypertrophying chondrocytes displayed very strong staining, particularly in their pericellular compartment (FIG. 1H). Boxed areas in FIGS. 1A and 1E are shown at higher magnification in FIGS. 1B and 1F, respectively. rc/pl, resting-proliferating cartilage; phc, pre-hypertrophic cartilage; hc, hypertrophic cartilage; pc, perichondrium. Scale bars in FIGS. 1A, 1B, 1D, 1E and 1H=250 μm, and in FIGS. 1B, 1F, and 1G=100 μm.

FIGS. 2A-2F show that treatment with human recombinant heparanase stimulates cell proliferation, migration and chondrogenesis. FIG. 2A provides DNA quantification showing that heparanase stimulates proliferation of ATDC5 cells in monolayer as early as 3 days after treatment when compared to vehicle-treated controls (n=3, p<0.05). FIG. 2B provides scratch wound-healing assays showing that cell migration is increased by heparanase treatment. Wound closure was monitored by sequential phase contrast imaging up to 3 hours (n=6, p<0.01). FIG. 2C provides representative images of alcian blue-stained limb bud cell micromass cultures on day 4 and 6 treated with recombinant heparanase or vehicle. Note the substantial increase in the number and staining intensity of cartilage nodules in treated versus control cultures. FIG. 2D provides image-based quantification of alcian blue positive areas showing a over 2-fold increase in treated cultures (n=6, p<0.01). FIG. 2E provides measurement of overall micromass diameter on day 4 and 6 indicates that proliferation/migration of peripheral cells was stimulated by heparanase treatment. Increase in diameter for each micromass was set relative to that at 24 hours after plating (n=6, p<0.01). FIG. 2F provides immunoblot images showing that heparanase treatment had increased Smad1/5/8 phosphorylation protein levels (pSmad) in day 4 and 6 micromass cultures. Membranes were re-blotted with α-tubulin antibodies (α-tub) for sample loading normalization.

FIGS. 3A-3H show heparanase gene expression is responsive to modulation in HS levels or function. FIG. 3A-3D provide immunoblot images and densitometric histograms indicating that endogenous heparanase protein levels were increased by Surfen treatment (indicated by a + sign) in limb bud micromasses (FIGS. 3A-3B) and ATDC5 cell cultures compared to respective untreated controls (indicated by a − sign) (FIGS. 3C-3D). FIGS. 3E-3F provide semi-quantitative RT-PCR and densitometric analyses showing that Surfen treatment increased heparanase gene expression in micromass cultures. FIGS. 3G-3H provide immunoblot and densitometric quantification analyses showing that endogenous heparanase protein levels were up-regulated by treatment with recombinant BMP2. Graphs depict means±S.D. Asterisk indicates statistical significance compared to control.

FIGS. 4A-4G show that chondrogenesis is inhibited by a heparanase antagonist. FIG. 4A provides images of alcian blue-stained micromass cultures on day 4 and 6 (left 6 panels) showing that treatment with the heparanase antagonist SST0001 (roneparstat) markedly reduced cartilage nodule formation and did so dose-dependently. The two panels on the right show images of control and treated micromass cultures counterstained with hematoxylin. FIG. 4B shows image-assisted quantification of alcian blue positive area in control versus SST0001(roneparstat)-treated cultures (n=6, p<0.01). Statistical significance was reached at every concentration tested. FIGS. 4C-4E provide histograms depicting gene expression levels of chondrogenic genes collagen II (ColII), aggrecan (Agg) and Runx2 in controls versus SST0001(roneparstat)-treated micromass cultures on day 4 and 6 measured by semi-quantitative RT-PCR and quantified by imaging (n=2). FIG. 4F provides immunoblot images showing that endogenous heparanase protein levels (Hep′ase) in micromass cultures were decreased by SST0001 (roneparstat) treatment (central lane) and increased by bacterial heparitinase (Hep) treatment (right lane) compared to control (left lane). FIG. 4G provides histograms depicting the overall micromass diameter at day 4 and 6 in control versus SST0001(roneparstat)-treated cultures (n=6, p<0.05). Graphs depict means±S.D.

FIG. 5 provides a schematic illustrating a series of regulatory steps that would cause inception and promotion of exostosis formation and growth. Point A: The cells of most HME patients bear a heterozygous loss-of-function mutation in EXT1 or EXT2 (depicted here at Ext+/− cells for simplicity). Point B: At one point pre-natally or post-natally, some cells would undergo a second genetic change (depicted here as fully colored cells along perichondrium and called “mutant”), resulting in a more severe loss of overall EXT expression or function and leading to further reduction in local HS production and levels. Point C: This in turn would cause an upregulation of heparanase (Hep′ase) expression, increases in growth factor availability and cell proliferation, and induction of ectopic chondrogenesis near/at perichondrium. Point D: The incipient exostosis cells would recruit surrounding heterozygous cells, induce them into neoplastic behavior and promote their incorporation into the outgrowing exostosis. Point E: Cells within the outgrowing exostosis would assemble into a typical growth plate-like structure protruding away from the surface of the skeletal element (depicted here as a long bone) and covered distally by perichondrium.

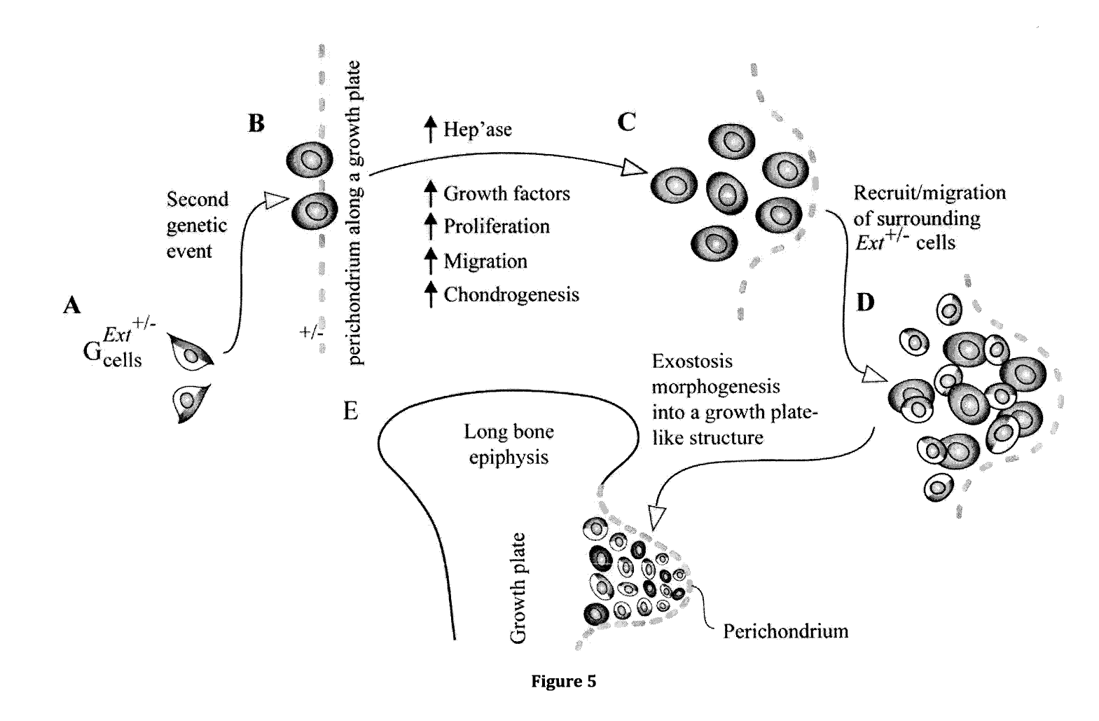

### DETAILED DESCRIPTION OF THE INVENTION

Hereditary Multiple Exostoses (HME) is characterized by ectopic growth plate-like benign tumors called exostoses that form next to growing skeletal elements and can cause multiple health problems. HME patients carry heterozygous mutations in the heparan sulfate (HS)-synthesizing enzymes EXT1 or EXT2, but studies suggest that EXT haploinsufficiency and ensuing partial HS deficiency are insufficient for exostosis formation. Herein, the presence and distribution of heparanase were examined in human exostoses from patients undergoing surgical treatment. Immunostaining showed that the protein was readily detectable in most chondrocytes, particularly in clusters of cells. In control human growth plates from unaffected individuals however, heparanase was detectable only in hypertrophic zone. Treatment of mouse embryo limb mesenchymal cells in micromass culture with exogenous recombinant human heparanase greatly stimulated chondrogenesis and BMP signaling as revealed by Smad1/5/8 phosphorylation. It also stimulated cell migration and proliferation in cell monolayers. Interfering with HS function with the chemical antagonist Surfen or treatment with bacterial heparitinase both up-regulated endogenous heparanase gene expression in chondrogenic cells, indicating a counterintuitive feedback mechanism that would result in further HS reduction and increased signaling. Thus, a potent heparanase inhibitor (roneparstat) was tested and it was found to strongly inhibit chondrogenesis. The data indicate that heparanase participates and is an important culprit in HME. Its widespread expression in exostoses and its ability to stimulate chondrogenesis, BMP signaling, cell migration and cell proliferation may all be causally linked to exostosis genesis and pathogenesis. Targeting heparanase thus represents a therapeutic opportunity to inhibit exostosis formation and limit the often severe skeletal and non-skeletal problems caused by the exostoses in children with HME.

Herein, it is shown that heparanase is detectable and broadly distributed in all the chondrocytes present in benign human exostoses, a phenotype quite distinct from that seen in normal growth plate cartilage where the protein is restricted to the hypertrophic zone and perichondrium (Trebicz-Geffen et al. (2008) Int. J. Exp. Path., 89:321-31). It is also shown that exogenous recombinant human heparanase is a strong stimulator of cell migration, proliferation and chondrogenesis in mouse cell cultures, indicating that one function—and possibly a main function—of heparanase would be to promote cell recruitment and the initiation and outgrowth of the exostoses. This mechanistic sequel is reinforced by the observations that heparanase stimulates the pro-chondrogenic BMP signaling pathway and that endogenous heparanase gene expression and overall protein levels are increased as the HS levels decrease. Thus, heparanase appears to be part of tightly controlled mechanisms linking it to HS levels and also responsive to HS level modulations. A significant drop in HS levels occurring in local cells in HME patients would elicit increased heparanase expression that in turn, would elicit further HS degradation, stimulate growth factor release and signaling pathways, and promote chondrogenesis, all steps converging on and contributing to exostosis growth. Although counterintuitive and seemingly paradoxical, similar inverse relationships amongst HS levels, Ext1 expression and heparanase expression have been demonstrated in cancer studies. Cancer cells with higher Ext1 expression exhibited lower heparanase expression while cancer cells with lower Ext1 expression (and higher metastatic potential) exhibited higher heparanase expression, and Ext1 knockdown with siRNA upregulated heparanase expression and potentiated metastatic capacity (Wang et al. (2013) Mol. Cell Biochem., 373:63-72). Indeed, heparanase expression levels are used to help predict tumor severity and patient outcome (Ilan et al. (2006) Int. J. Biochem. Cell B., 38:2018-2039; Quiros et al. (2006) Cancer 106:532-540; Vlodaysky et al. (2001) J. Clin. Invest., 108:341-347). Thus, one of the changes occurring during the transition from benign exostoses to osteosarcoma in HME patients could be an even steeper increase in heparanase expression.

As indicated above, EXT haploinsufficiency per se is not sufficient for multiple exostosis formation in patients or mice and that a steeper reduction would be needed (Reijnders et al. (2010) Am. J. Path., 177:1946-57; Stickens et al. (2005) Development. 132:5055-68). Current possible and plausible explanations of how Ext expression and/or HS production could decrease further include: EXT loss-of-heterozygosity; large genomic deletions; a second hit in another gene; background genetic variability including gene modifiers; or compound heterozygosity in EXT1 and EXT2 (Jennes et al. (2009) Hum. Mutat., 30:1620-7; Waaijer et al. (2013) Genes Chromosomes Cancer 52:431-6; Wuyts et al. (2000) Hum. Mutat., 15:220-7; Pedrini et al. (2011) J. Bone Joint Surg., 93:2294-2302). Such changes would occur in subsets of cells within the HME patient; the cells would be prone to exostosis formation and would give rise to exostoses in conjunction with an active growth plate. The data provided herein shows that heparanase also plays a role in such pathogenic scenarios. The protein can contribute to disease regardless of which of the various genetic processes above leads to a steeper decrease in EXT expression and HS levels below those achieved by mere haploinsufficiency. Notably, heparanase is preferentially expressed in perichondrium in normal control growth plates. Similar data has been seen in the mouse growth plate perichondrium (Brown et al. (2008) Bone 43:689-99). Further, progenitor cells located within perichondrium may be a primary contributor to exostosis formation (Huegel et al. (2013) Dev. Biol., 377:100-12). Thus, by possessing basal heparanase expression, perichondrial cells may be particularly sensitive to the above genetic changes and could require a lower threshold to tilt their homeostatic balance, boost basal heparanase expression, incite ectopic chondrogenesis, and promote exostosis formation. One additional aspect of exostosis formation stemming from mouse and human studies is that the exostoses themselves appear to be composed by a mixture of mutant cells (that is, heterozygous Ext cells that have undergone an additional genetic change above) and plain heterozygous cells (Huegel et al. (2013) Dev. Biol., 377:100-12; Reijnders et al. (2010) Am. J. Path., 177:1946-57). The mutant cells would recruit the heterozygous cells into the incipient exostosis mass and induce them into a neoplastic behavior. Given that heparanase facilitates cell migration and could diffuse into the surroundings, it can have a role in such recruitment and mobilization process during exostosis growth.

The heparanase inhibitor roneparstat has been shown to interfere with the growth of myeloma and sarcoma cells and concomitant angiogenesis in mouse models (Ritchie et al. (2011) Clin. Cancer Res., 17:1382-1393; Cassinelli et al. (2013) Biochem. Pharmacol., 85:1424-1432), reaffirming that heparanase has a major role in pathogenesis and may be a particularly good target for cancer therapy. The data provided herein with primary limb cell micromass cultures provide evidence that roneparstat also inhibits chondrogenesis and does so powerfully as revealed by major decreases in development of alcian blue-positive cartilage nodules and expression of such chondrogenic marker genes as collagen II, aggrecan and Runx2. A different heparanase inhibitor—PI-88—was used with the ADTC5 chondrogenic cell line in which only modest reductions in alcian blue staining and cartilage gene expression were observed (Brown et al. (2008) Bone 43:689-99). The data provided herein demonstrate that heparanase has a far more important role in chondrogenesis than previously realized and may be essential in mobilizing chondrogenic factors and enhancing their bioavailability and diffusion amongst condensed prechondrogenic cells. Its effective suppression would hamper this process and elicit a strong anti-chondrogenic effect. Similarly, roneparstat may interfere with cell mobilization and cell-cell interactions needed by the limb mesenchymal cells to initially condense and then activate the chondrogenic program (Ahrens et al. (1979) Dev. Biol., 69:436-50). Roneparstat is a heparin modified through N-desulfation, subsequent N-reacetylation and glycol splitting (Naggi et al. (2005) J. Biol. Chem., 280:12103-121), which is no longer anticoagulant and cannot dislodge basic fibroblast growth factor from the extracellular matrix and enhance its mitogenic activity, traits that native heparins and HS chains have (Bernfield et al. (1999) Annu. Rev. Biochem., 68:729-777; Whitelock et al. (2005) Chem. Rev., 105:2745-2764). However, by still being a partially sulfated polymer, roneparstat could bind other growth factors including BMPs and limit their bioactivity. The data provided herein also show that it decreased the endogenous levels of heparanase in the micromasses and reduced cell migration. Thus, the data indicate that heparanase inhibitors, particularly modified heparins such as roneparstat, are potent inhibitor of exostosis formation in vivo.

The instant invention encompasses methods of inhibiting and/or preventing chondrogenesis (particularly aberrant, excessive, and/or improper chondrogenesis) and/or exostosis formation. The instant invention also encompasses methods of treating, inhibiting, and/or preventing diseases or disorders associated with aberrant, excessive, or improper chondrogenesis and/or exostosis formation. Examples of such chondrogenesis or exostosis related diseases and disorders include, without limitation: hereditary multiple exostoses (HME; also known as multiple hereditary exostoses, diaphyseal aclasis, and multiple osteochondromatosis), congenital conditions such as metachondromatosis (characterized by both exostoses and enchondromas), fibrodysplasia ossificans progressiva (characterized by heterotopic ossification and exostoses), ectopic chondrogenesis (e.g., during osteophyte formation in osteoarthritis), chondrogenic transdifferentiation (e.g., in tendons after rapture/damage), and ectopic chondrogenesis in perispinal ligaments and/or arteries such as aorta (e.g., occurring in PPi deficiency or in patients taking warfarin or other anticoagulants; see, e.g., Johnson et al. (2005) Arterioscler. Thromb. Vasc. Biol., 25:686-691). The methods of the instant invention comprise administering at least one heparanase inhibitor to a subject in need thereof.

In a particular embodiment, the heparanase inhibitor of the instant invention is selected from the group consisting of heparin, a modified heparin, antibodies (e.g., neutralizing antibodies), small compounds/drugs that block enzymatic activity, and inhibitory nucleic acids (e.g., RNA interference, microRNA, siRNA, antisense, etc.) to block heparanase gene expression. Particularly, the heparanase inhibitor is a modified heparin, particularly one which is modified through glycol splitting, partial desulfation, and/or N-acetylation. In a particular embodiment, the modified heparin is no longer an anticoagulant (e.g., the heparin has lost at least about 80%, about 90% or more of its anticoagulation activity). Examples of modified heparins are provided in Naggi et al. (J. Biol. Chem. (2005) 280:12103-121). In a particular embodiment, the modified heparin is a modified heparin from Table 1 of Naggi et al. (J. Biol. Chem. (2005) 280:12103-121) including, without limitation: RO—H; ²⁶NA,RO—H; ⁴⁰NA,RO—H; ⁵³NA,RO—H; ⁶⁷NA,RO—H; ¹⁰⁰NA,RO—H; ⁷⁰NAH, ⁵⁹gs; and LMHW, ⁵⁰NA,gsRO—H. Further examples of heparanase inhibitors include, without limitation PG545, M402, and PI-88 (see, e.g., Hammond et al. (2012) PLoS One, 7(12): e52175; Zhou et al. (2011) PLoS One, 6(6):e21106; and Progen Pharmaceuticals Ltd., Brisbane, QLD, Australia).

In a particular embodiment, the modified heparin is SST0001 (roneparstat). Roneparstat (also designated as ¹⁰⁰NA,RO—H or SST0001 or G4000) is a modified heparin derivative that is N-desulphated (e.g., 100%), N-reacetylated and glycol split (Casu et al. (2008) Pathophysiol. Haemost. Thromb. 36:195-20; Naggi et al. (2005) J. Biol. Chem., 280:12103-13). These modifications abolish the anticoagulant activity, but enhance the inhibition of heparanase. Roneparstat shows antiangiogenic activity and efficacy in preclinical models of cancers and has recently entered Phase I clinical trials in patients with multiple myeloma. Roneparstat also markedly decreases the extent of albuminuria and renal damage in mouse models of diabetic nephropathy.

In a particular embodiment, the modified heparin is a glycol split derivative. In a particular embodiment, the glycol splitting comprises cleaving the C-2-C-3 bonds of GlcA or nonsulfated uronic acid residues. Without being bound by theory, it is believed that the glycol splitting provides flexibility to the heparin and reduces heparin's anticoagulation activity. Glycol splitting may be performed by periodate oxidation/borohydride reduction of heparin (see, e.g., Naggi et al. (2005) J. Biol. Chem., 280:12103-121). In a particular embodiment, the heparin is less than about 75% glycol split or less than about 60% glycol split. In a particular embodiment, the heparin is about 1% to about 100% glycol split, 1% to about 75% glycol split, about 1% to about 60% glycol split, about 1% to about 50% glycol split, about 5% to about 40% glycol split, about 10% to about 40% glycol split, about 15% to about 35% glycol split, about 20% to about 30% glycol split, or about 25% glycol split.

In a particular embodiment, the modified heparin is desulfated or partially desulfated. The heparin may be 6-O-desulfated and/or 2-O-desulfated. In a particular embodiment, the 6-O-sulfate of a glucosamine residue and/or the 2-O-sulfate of an iduronic acid residue is removed. In a particular embodiment, the desulfation does not result in a conformational change of IdoA to GalA. In a particular embodiment, the heparin is about 1% to about 100% desulfated or about 1% to about 80% desulfated. In a particular embodiment, the heparin is at least about 90%, 95%, 97%, 99%, or 99.5% desulfated.

In a particular embodiment, the modified heparin is N-acetylated. In other words, the N-sulfate groups of heparin may be replaced with N-acetyl groups. In a particular embodiment, the N-acetylation is from about 1% to about 100%, about 10% to about 90%, about 25% to about 75%, about 30% to about 70%, about 30% to about 50%, or about 40%. In a particular embodiment, the N-acetylation is at least about 90%, 95%, 97%, 99%, or 99.5%.

The heparin or modified heparin of the present invention may be prepared in a variety of ways, according to known methods. For example, heparin may be purified from appropriate sources, e.g., mammals, including humans.

In a particular embodiment of the present invention, the heparanase inhibitor(s) of the instant invention may be administered to a patient in a pharmaceutically acceptable carrier, particularly via injection. The heparanase inhibitor of the instant invention may optionally be encapsulated into liposomes or mixed with other phospholipids or micelles to increase stability of the molecule. The heparanase inhibitor may be administered alone or in combination with other agents known to inhibit chondrogenesis or exostosis formation or treat, inhibit, and/or prevent diseases or disorders associated with improper chondrogenesis or exostosis formation (e.g., another anti-chondrogenic agent such as a synthetic retinoid agonist). The compounds may be contained in the same composition or may be in a separate composition. The compositions may be administered concurrently or consecutively.

The heparanase inhibitor(s) (or composition(s) comprising the same) can be administered by any suitable route, for example, by injection (e.g., for local, direct, or systemic administration), oral, pulmonary, topical, nasal or other modes of administration. The composition may be administered by any suitable means, including parenteral, intramuscular, intravenous, intravascular, intraarterial, intraperitoneal, subcutaneous, topical, inhalatory, transdermal, intrapulmonary, intraareterial, intrarectal, intramuscular, and intranasal administration. In a particular embodiment, the composition is administered subcutaneously. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences and Remington: The Science and Practice of Pharmacy). The pharmaceutical composition of the present invention can be prepared, for example, in liquid form, or can be in dried powder form (e.g., lyophilized for later reconstitution).

The therapeutic agents described herein will generally be administered to a subject as a pharmaceutical preparation. The compositions of the instant invention may be employed therapeutically or prophylactically, under the guidance of a physician.

The compositions comprising the heparanase inhibitor of the instant invention may be conveniently formulated for administration with any pharmaceutically acceptable carrier(s). The concentration of agent in the chosen medium may be varied and the medium may be chosen based on the desired route of administration of the pharmaceutical preparation. Except insofar as any conventional media or agent is incompatible with the agent to be administered, its use in the pharmaceutical preparation is contemplated.

The dose and dosage regimen of the agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation or prevention of a particular condition may be determined by dosage concentration curve calculations, as known in the art.

The pharmaceutical preparation comprising the agent may be administered at appropriate intervals until the pathological symptoms are reduced or alleviated, after which the dosage may be reduced to a maintenance level. The appropriate interval in a particular case would normally depend on the condition of the patient. Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a therapeutically or prophylactically effective amount.

### Definitions

The following definitions are provided to facilitate an understanding of the present invention:

The singular forms “a,” “an,” and “the” include plural referents unless the context clearly dictates otherwise.

As used herein, the terms “host,” “subject,” and “patient” refer to any animal, particularly mammals including humans.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., HME) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to modulate chondrogenesis or exostosis formation in a subject.

The following example is provided to illustrate various embodiments of the present invention. The example is illustrative and is not intended to limit the invention in any way.

### EXAMPLE

### Materials and Methods

### Human Exostosis Samples

Exostosis tissue samples from four consenting HME patients were taken after surgical removal and were processed by the centralized Children's Hospital of Philadelphia (CHOP) pathology laboratory. Control growth plate tissue was harvested from surgically removed toes of children who were undergoing treatment for polydactyly. Fixed tissue was embedded in paraffin and sectioned (6 μm). After specimens were used for clinical diagnosis, extra slides were de-identified and provided for the present study. This practice was reviewed and approved by CHOP's Institutional Review Board.

### Monolayer and Micromass Cell Culture

ATDC5 cells, derived from mouse embryonal carcinoma, were cultured in 10 cm dishes with a 1:1 mixture of Dulbecco's modified Eagle's medium and Ham's F-12 (Life Technologies) supplemented with 5% fetal bovine serum (ATCC), 10 μg/ml human transferrin (Mediatech), 3×10⁴ mg/L sodium selenite (Mediatech), and 20 μg/ml insulin at 37° C. under 5% CO₂. Experiments were conducted once cells reached confluence.

Micromass cultures were prepared from the mesenchymal cells of E11.5 mouse embryo limb buds (Ahrens et al. (1979) Dev. Biol., 69:436-50). Dissociated cells were suspended at a concentration of 5×10⁶ cells/ml in DMEM containing 3% fetal bovine serum and antibiotics. Micromass cultures were initiated by spotting 20 μl of the cell suspensions (1.5×10⁵ cells) onto the surface of 24-well tissue culture dishes. After a 90-minute incubation at 37° C. in a humidified CO₂ incubator to allow for cell attachment, the cultures were supplied with 0.25 ml of medium. After 24 hours, medium supplemented with the indicated concentrations of Surfen (bis-2-methyl-4-aminoquinolyl-6-carbamide), bacterial heparitinase III (Sigma), recombinant human BMP2 (R&D Systems), recombinant human heparanase, or roneparstat (Sigma-Tau Research S.A.). Fresh reagents (drug and/or protein) were given with medium change every other day. Equivalent amounts of vehicle were added to control cultures. Cultures were stained with Alcian blue (pH 1.0) after four and six days to monitor chondrogenic cell differentiation by standard methods. Micromass analysis was performed using ImageJ. Images were made binary under an RGB threshold, and “Particle Analysis” was utilized to measure nodule size, number, and Alcian blue positive area.

### Cell Proliferation and Migration Analysis

ATDC5 cells were treated at day 0 with human heparanase (400 ng/ml) or a vehicle control. At this time, one culture dish was harvested for DNA. DNA was also isolated from control and treated cells at day 3, 7, and 14. DNA isolation was performed using TRIzol® reagent (Invitrogen) according to the manufacturer's protocols and measured using a NanoDrop (ThermoScientific). ATDC5 migration was analyzed via a scratch test. Cells were replated on a six-well dish and allowed to reach confluence before treatment. After 24 hours of treatment, a straight line “scratch” was made using a P10 pipette tip. Reference points were made with a fine-tipped marker on the bottom of the dish. Images at time 0 were taken with a phase-contrast microscope (Leica). Cells were cultured at 37° C. in a humidified CO₂ incubator for four hours, with images in the same field of view taken at 2 and 4 hours. The open scratch area was quantified with ImageJ (n=6/treatment group). In micromass cultures, migration was determined by expansion of the micromass itself from day 0 to day 4 or 6. The diameters of untreated micromasses (n=10) were measured and averaged for a baseline size. This baseline was subtracted from the average diameter of treated cultures (n=6) to estimate cell migration.

### Semi-Quantitative PCR Analyses

Total RNA was extracted from cells by the guanidine phenol method using TRIzol® reagent (Invitrogen) according to the manufacturer's protocols and measured using a NanoDrop. One μg of total RNA was reverse transcribed using the SuperScript® III First-Strand Synthesis System (Life Technologies). Band intensities for semiquantitative PCR were normalized to GAPDH and compared while band intensities and cycle numbers were linear. The following primer sets were used: Gapdh forward primer (5′-CGTCCCGTAGACAAAATGGT-3′; SEQ ID NO: 1) and reverse primer (5′-TTGATGGCAACAATCTCCAC-3′; SEQ ID NO: 2); Runx2 forward primer (5′-CGCACGACAACCGCACCAT-3′; SEQ ID NO: 3) and reverse primer 5′-AACTTCCTGTGCTCCGTGCTG-3′; SEQ ID NO: 4); Agg forward primer (5′-GGAGCGAGTCCAACTCTTCA-3′; SEQ ID NO: 5) and reverse primer (5′-CGCTCAGTGAGTTGTCATGG-3′; SEQ ID NO: 6); Col2a1 forward primer (5′-CTACGGTGTCAGGGCCAG-3′; SEQ ID NO: 7) and reverse primer (5′-GTGTCACACACACAGATGCG-3′; SEQ ID NO: 8); BMP2 forward primer (5′-TCTTCCGGGAACAGATACAGG-3′; SEQ ID NO: 9) and reverse primer (5′-TCTCCTCTAAATGGGCCACTT-3′; SEQ ID NO: 10); BMPRI forward primer (5′-GCTTGCGGCAATCGTGTCTAA-3′; SEQ ID NO: 11) and reverse primer (5′-GCAGCCTGTGAAGATGTAGAGG-3′; SEQ ID NO: 12); BMPRII forward primer (5′-CACACCAGCCTTATACTCTAGATA-3′; SEQ ID NO: 13) and reverse primer (5′-CACATATCTGTTATGAAACTTGAG-3′; SEQ ID NO: 14). Band intensities were quantified by computer-assisted image analysis (ImageQuant™ TL, GE Healthcare).

### Protein Analysis

ATDC5 cells were grown to 100% confluence in 6-well plates and treated with indicated concentrations of Surfen, bis-2-methyl-4-amino-quinolyl-6-carbamide. Micromass cultures were treated with control vehicle, Surfen, human heparanase, roneparstat, or rhBMP2. At specified time points, total cellular proteins were harvested in SDS-PAGE sample buffer, electrophoresed on 4-15% SDS-Bis-Tris gels (40 μg per lane) and transferred to PVDF membranes (Invitrogen). Membranes were incubated overnight at 4° C. with dilutions of antibodies against phospho-Smad1/5/8 (Cell Signaling Technology 9511, 1:1000), Smad1 (Abcam 63439, 1:500) or human heparanase (Abcam 59787, 1:750). Enhanced chemiluminescent immunoblotting detection system (Pierce) was used to detect the antigen antibody complexes. The membranes were re-blotted with antibodies to α-tubulin (Sigma T-5168, 1:2000) for normalization, and band intensities were quantified by computer-assisted image analysis.

### Immunohistochemistry

Immunostaining for heparanase was carried out with paraffin sections that were first deparaffinized and then treated with 5 mg/ml hyaluronidase for one hour at 37° C. to de-mask the tissue. Sections were incubated with anti-heparanase polyclonal antibodies (Abeam 59787) at 1:200 dilution in 3% NGS in PBS overnight at 4° C. Following rinsing, sections were then incubated with biotinylated anti-rabbit secondary antibody, and the signal was visualized using a HRP/DAB detection IHC kit according to the manufacturer's instructions (Abeam). Bright-field images were taken with a SPOT insight camera (Diagnostic Instruments, Inc.) operated with SPOT 4.0 software.

### Statistical Analyses

Data were validated by two-tailed Student's t-tests. The threshold for significance for all tests was set as p<0.05.

### Results

### Broad Heparanase Distribution in Human Exostoses

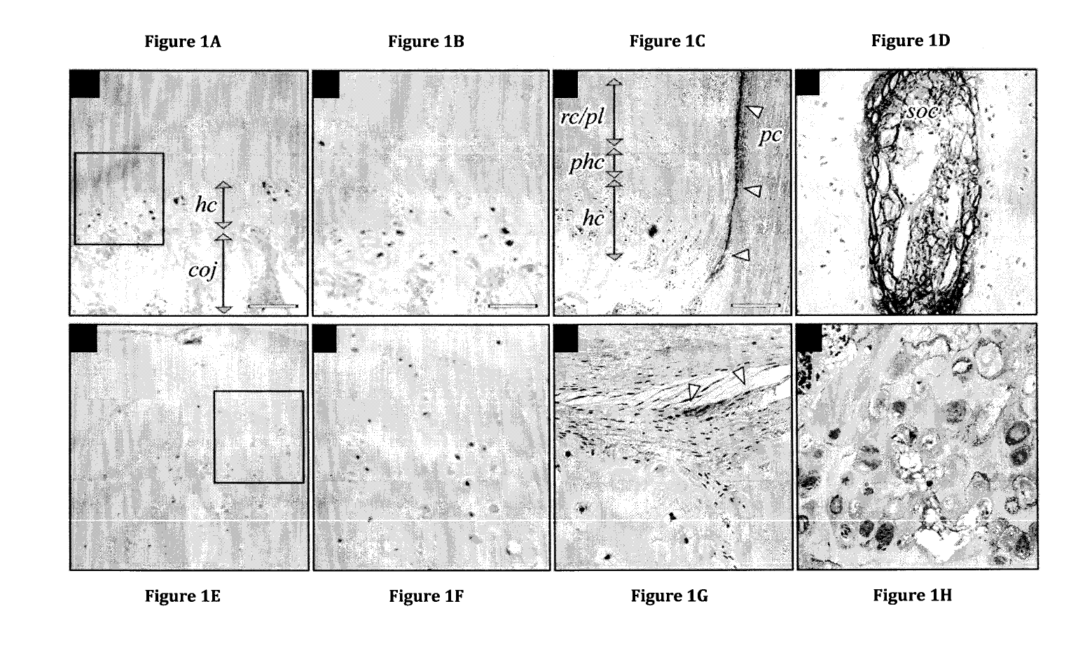

Mature and symptomatic exostoses removed at surgery are typically composed of a cartilaginous cap and an underlying bony stem that connects with the affected skeletal element, be it a long bone or a rib. The cartilaginous cap often displays a growth plate-like structure and organization, with small chondrocytes located at its distal end and surrounded by a connective/perichondrium-like tissue layer and with large hypertrophying chondrocytes located more proximally near the bony portion (Jones, K. B. (2011) J. Pediatr. Orthop., 31:577-86; Porter et al. (1999) J. Pathol., 188:119-25). To determine whether heparanase presence and distribution were altered in exostoses, exostoses were obtained from consenting HME patients undergoing surgical treatment and processed them for section immunostaining with rabbit antibodies against human heparanase. For comparison, longitudinal sections of growth plates from control non-affected individuals who had undergone surgical treatment for polydactyly were used. In these control specimens, heparanase staining was clearly and strongly detected in the hypertrophic chondrocytes at the chondro-osseous junction (FIGS. 1A-1B) and in the perichondrium (FIG. 1C, arrowheads), but little to no staining was observed in resting and proliferative zones (FIG. 1A-1C). Blood vessels invading the secondary ossification center displayed very high heparanase staining as expected (Elkin et al. (2001) FASEB J., 15:1661-3), thus acting as an internal positive control of staining specificity and sensitivity (FIG. 1D). In contrast, there was heparanase staining in all chondrocytes within the exostoses, regardless of location within the tissue and apparent maturation stage (FIGS. 1E-1G). Tissue portions containing enlarged and hypertrophying cells in clusters were even more strongly stained, particularly in their pericellular compartment (FIG. 1H).

### Heparanase Stimulates Cell Migration and Proliferation and Chondrogenesis

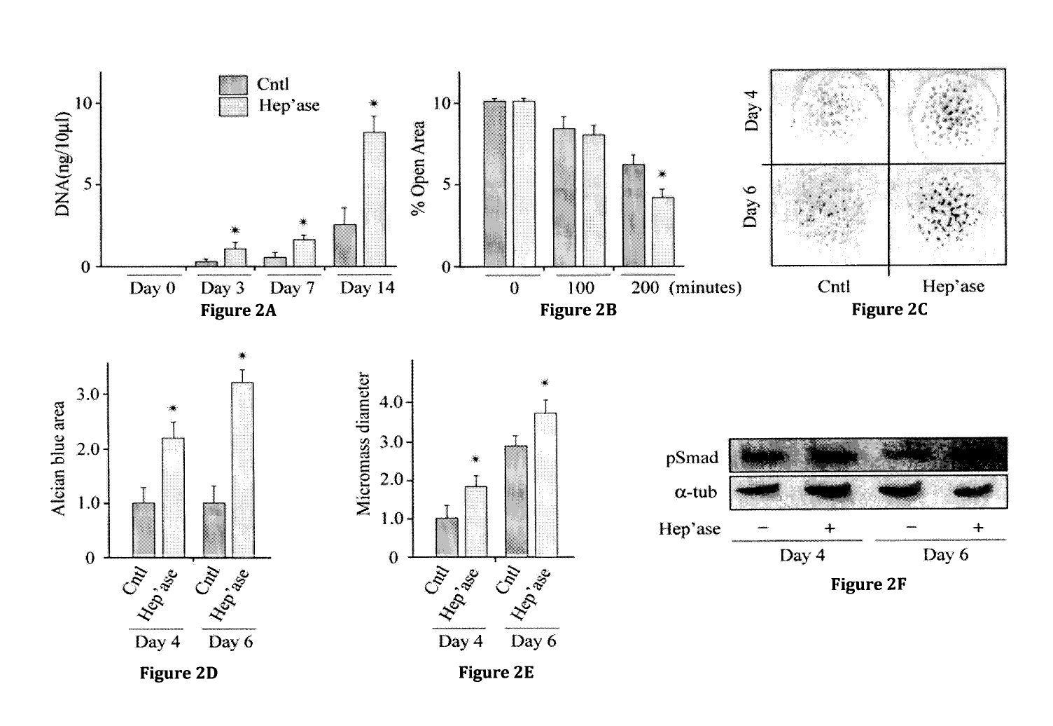

Exostoses are ectopic cartilaginous outgrowths and as such, must depend on local cell proliferation and migration and on chondrogenic differentiation to initiate, sustain and propel their development and growth (Huegel et al. (2013) Dev. Dyn., 242:1021-32). Thus, it was investigated whether heparanase would influence such processes. To analyze cells proliferation, ATDC5 chondrogenic cells were reared in monolayer culture in control medium or medium containing 400 ng/ml human recombinant heparanase, a concentration used in previous studies and eliciting maximal responses (Gingis-Velitski et al. (2004) J. Biol. Chem., 279:23536-41). DNA quantification at 3 time points over a 2 week culture period showed that heparanase had in fact stimulated cell growth by nearly 3 fold by day 14 over control values (FIG. 2A). To examine cell migration, the scratch and “wound healing” system was used (Gingis-Velitski et al. (2004) J. Biol. Chem., 279:23536-41). Accordingly, confluent monolayers were scratched in the middle with a pipette, and the repopulation of the scratch area by migrating cells was determined over a 3-hour period in the absence or presence of exogenous heparanase. Clearly, cultures treated with heparanase repopulated the wound area more rapidly and more extensively than untreated control cells by 3 hours (FIG. 2B).

To examine chondrogenic cell differentiation, micromass cultures were utilized (Ahrens, P. B. (1979) Dev. Biol., 69:436-50; Huegel et al. (2013) Dev. Biol., 377:100-12). In this popular experimental system, progenitor chondrogenic cells are isolated from embryonic limb buds and are grown at high cell density in spot cultures where they resume their differentiation program and give rise to cartilaginous nodules over time. For these experiments, cells isolated from mouse embryo limb buds were plated in micromass and grown in medium lacking or containing exogenous heparanase as above. Staining with alcian blue on day 4 and 6 showed that control cultures displayed several positive cartilage nodules scattered in their central portion as expected (FIG. 2C, left panels), but more numerous and more strongly staining nodules were present in companion heparanase-treated cultures (FIG. 2C, right panels). Computer-assisted imaging showed that the alcian blue-staining area was over 2 fold higher in treated than control cultures (FIG. 2D, p<0.05). Similar results were obtained using exogenous bacterial heparitinase III that cleaves HS in far smaller fragments than mammalian enzyme (Parish et al. (2001) Biochim. Biophys. Acta., 1471:M99-108), indicating that chondrogenesis is stimulated regardless of HS fragment size. The peripheral portion of micromass cultures normally contain variously shaped fibroblastic cells that actively proliferate and migrate away from the micromass over time (Ahrens et al. (1979) Dev. Biol., 69:436-50). Thus, the overall maximal diameter of the cultures was measured and it was found that the heparanase-treated cultures had achieved a significantly larger diameter at both day 4 and day 6 compared to controls (FIG. 2E), reaffirming that heparanase promotes cell proliferation and/or migration.

BMP signaling is a major regulator and stimulator of chondrogenesis (Kronenberg, H. M. (2003) Nature 423: 332-336) and it is ectopically activated during the early stages of exostosis-like tissue formation in HME mouse models involving conditional Ext1 ablation (Parish et al. (2001) Biochim. Biophys. Acta., 1471:M99-108). Thus, it was tested whether human heparanase treatment would stimulate BMP signaling along with its stimulation of chondrogenesis as seen above. Immunoblot analysis with antibodies to phosphorylated Smad1/5/8 using whole micromass cell extracts showed that pSmad levels were in fact markedly increased after heparanase treatment at both day 4 and day 6 of culture compared to controls (FIG. 2F), likely signifying that heparanase had stimulated chondrogenesis by increasing bioavailability and/or signaling activity of endogenous cell/matrix-associated BMPs.

### Heparanase Gene Expression is Responsive to HS Levels

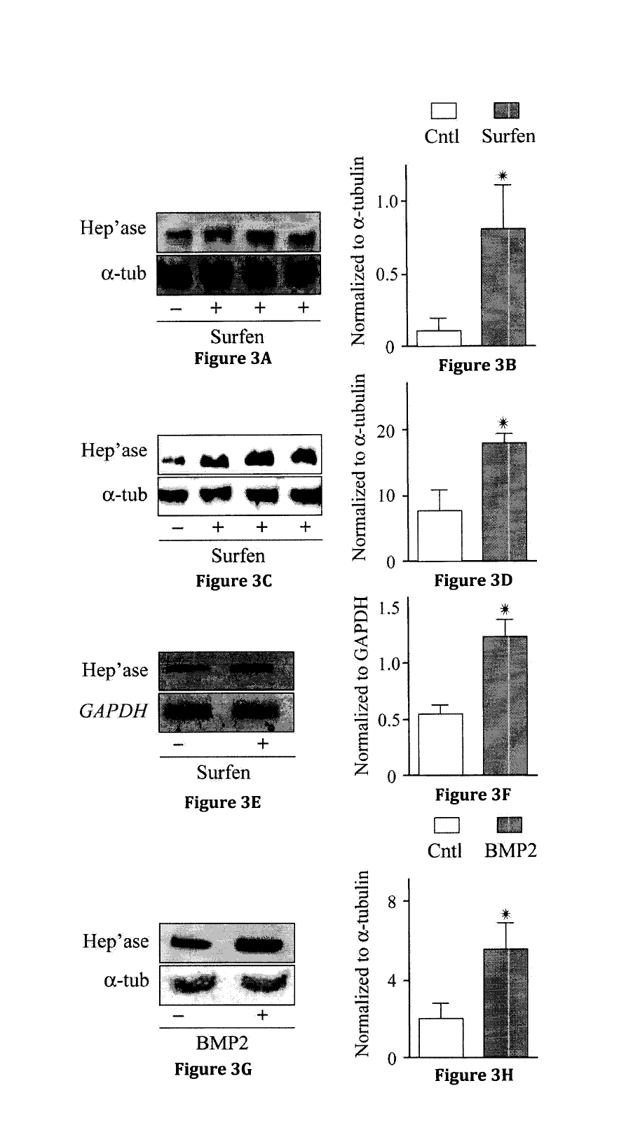

The widespread presence of heparanase in exostoses and its pro-chondrogenic and signaling effects above raised the question of whether heparanase is an early response gene, able to be up-regulated soon after or concomitantly with declining HS levels and thus perpetuating or amplifying cellular responses. To test this thesis, primary limb mesenchymal cell micromass cultures were treated with the HS antagonist Surfen. This drug-like compound interferes with HS function and elicits functional HS deficiency (Schuksz et al. (2008) Proc. Natl. Acad. Sci., 105:13075-80), triggering cellular responses similar to Ext gene ablation including enhanced chondrogenesis (Huegel et al. (2013) Dev. Biol., 377:100-12). Interestingly, it was found that treatment with an optimal concentration of Surfen (7.5 μM) (Huegel et al. (2013) Dev. Biol., 377:100-12) significantly increased the protein levels of endogenous heparanase as indicated by immunoblotting (FIG. 3A), about 8-fold at this dose (FIG. 3B). A very similar response was observed in ADTC5 cell cultures (FIG. 3C-3D). Control of this response appeared to be at the transcriptional and/or RNA stabilization levels since the heparanase mRNA amounts were increased as well in Surfen-treated cultures as indicated by semi-quantitative RT-PCR (FIG. 3E-3F).

A related and congruent possibility is that endogenous heparanase levels may be stimulated not only by declining HS levels/function, but also concurrent BMP bioavailability. Thus, day 2 micromass cultures were treated with exogenous 100 ng/ml rhBMP2, a concentration that strongly stimulates BMP signaling and chondrogenesis (Huegel et al. (2013) Dev. Biol., 377:100-12), for 24 or 48 hours. Immunoblot analysis showed that endogenous heparanase levels were clearly increased in BMP2-treated than companion untreated cultures (FIG. 3G), about 2 fold as revealed by densitometry and normalization (FIG. 3H).

### Chondrogenesis is Inhibited by a Heparanase Antagonist

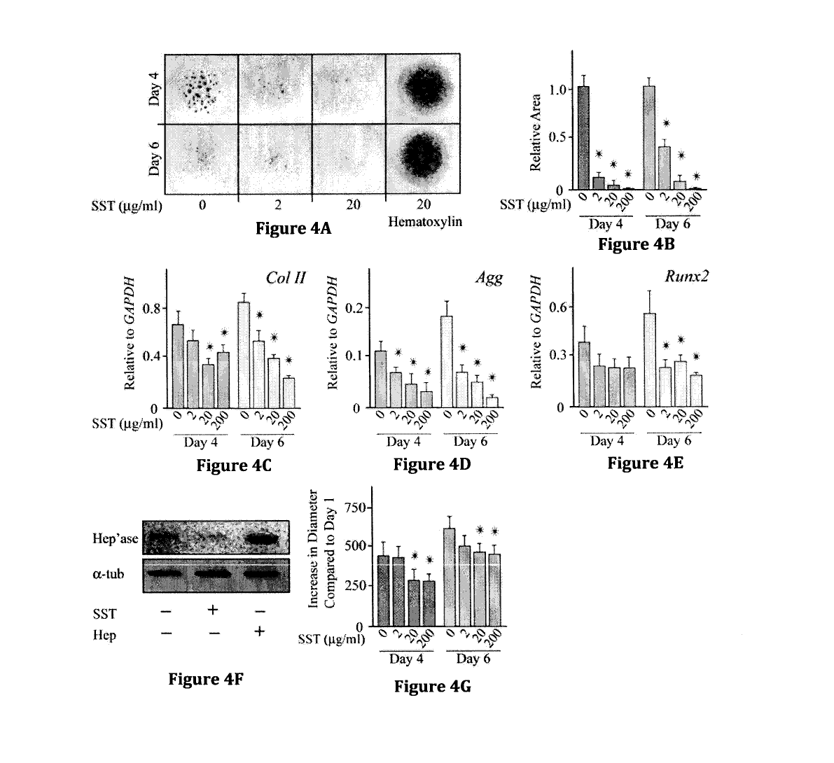

Given the above results linking heparanase to the stimulation of chondrogenesis and BMP signaling, we asked whether inhibiting endogenous heparanase would have negative repercussions on chondrogenesis. Thus, the heparanase inhibitor roneparstat, a modified heparin molecule without anticoagulant properties and with high affinity for heparanase and very strong inhibitory activity, was used (Ritchie et al. (2011) Clin. Cancer Res., 17:1382-1393). This compound has already been thoroughly tested for safety and pharmacokinetics in a number of animal models, and has been to shown to have anti-tumorigenic properties (Cassinelli et al. (2013) Biochem. Pharmacol., 85:1424-1432). Based on this groundwork, three concentrations were chosen with which to treat limb bud micromass cultures, at or above the IC₅₀ for heparanase inhibition. It was found that roneparstat treatment strongly reduced the formation of alcian blue-positive chondrogenic nodules (FIG. 4A, left side panels) in a dose-dependent manner as indicated by image quantification (FIG. 4B). Counterstaining with hematoxylin showed that overall cell number and density had not changed significantly after treatment (FIG. 4A, right side panels). Whole cellular RNA and DNA content also remained similar in treated and untreated cultures at day 4 and day 6, though minor decreases were observed by these methods at the maximal 20 μg/ml dose. Whole cellular RNA samples were then processed for semiquantitative RT-PCR, and the data showed that expression of such chondrogenic marker genes as collagen II (ColII), aggrecan (Agg) and Runx2 was dose-dependently inhibited by roneparstat treatment (FIG. 4C-4E). Interestingly, roneparstat treatment also reduced the levels of endogenous heparanase (FIG. 4F, central lane) compared to untreated control cultures (FIG. 4F, left lane), while treatment with bacterial heparitinase (Hep) increased them (FIG. 4F, right lane). As well, roneparstat treatment reduced the overall diameter of the micromasses (FIG. 4G) indicating that it had inhibited migration of peripheral fibroblastic cells.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for inhibiting, treating, and/or preventing a chondrogenesis-related disease or disorder in a subject, said method comprising administering to a subject at least one heparanase inhibitor.

2. The method of claim 1, wherein said chondrogenesis-related disease or disorder is hereditary multiple exostoses.

3. A method for inhibiting or preventing exostosis formation or growth in a subject, said method comprising administering to a subject at least one heparanase inhibitor.

4. The method of claim 3, wherein said heparanase inhibitor is a modified heparin.

5. The method of claim 4, wherein said modified heparin is glycol split heparin.

6. The method of claim 4, wherein said modified heparin is at least partially desulfated.

7. The method of claim 4, wherein said modified heparin is N-acetylated.

8. The method of claim 4, wherein said modified heparin is roneparstat.

9. The method of claim 4, wherein said modified heparin is glycol split heparin and at least partially desulfated.

10. The method of claim 4, wherein said modified heparin is N-acetylated.

11. The method of claim 4, wherein said modified heparin is glycol split heparin and N-acetylated.

12. The method of claim 4, wherein said modified heparin is glycol split heparin, N-acetylated, and at least partially desulfated.

13. The method of claim 1, wherein said heparanase inhibitor is a modified heparin.

14. The method of claim 13, wherein said modified heparin is glycol split heparin.

15. The method of claim 13, wherein said modified heparin is at least partially desulfated.

16. The method of claim 13, wherein said modified heparin is N-acetylated.

17. The method of claim 13, wherein said modified heparin is roneparstat.

18. The method of claim 13, wherein said modified heparin is glycol split heparin and at least partially desulfated.

19. The method of claim 13, wherein said modified heparin is N-acetylated.

20. The method of claim 13, wherein said modified heparin is glycol split heparin and N-acetylated.

21. The method of claim 13, wherein said modified heparin is glycol split heparin, N-acetylated, and at least partially desulfated.


SEQUENCE LISTING (14 sequences)

SEQ ID NO: 1 - DNA, 20 bp, Artificial Sequence
  Gapdh forward primer
cgtcccgtagacaaaatggt

SEQ ID NO: 2 - DNA, 20 bp, Artificial Sequence
  Gapdh reverse primer
ttgatggcaacaatctccac

SEQ ID NO: 3 - DNA, 19 bp, Artificial Sequence
  Runx2 forward primer
cgcacgacaaccgcaccat

SEQ ID NO: 4 - DNA, 21 bp, Artificial Sequence
  Runx2 reverse primer
aacttcctgtgctccgtgctg

SEQ ID NO: 5 - DNA, 20 bp, Artificial Sequence
  Agg forward primer
ggagcgagtccaactcttca

SEQ ID NO: 6 - DNA, 20 bp, Artificial Sequence
  Agg reverse primer
cgctcagtgagttgtcatgg

SEQ ID NO: 7 - DNA, 18 bp, Artificial Sequence
  Col2a1 forward primer
ctacggtgtcagggccag

SEQ ID NO: 8 - DNA, 20 bp, Artificial Sequence
  Col2a1 reverse primer
gtgtcacacacacagatgcg

SEQ ID NO: 9 - DNA, 21 bp, Artificial Sequence
  BMP2 forward primer
tcttccgggaacagatacagg

SEQ ID NO: 10 - DNA, 21 bp, Artificial Sequence
  BMP2 reverse primer
tctcctctaaatgggccactt

SEQ ID NO: 11 - DNA, 21 bp, Artificial Sequence
  BMPRI forward primer
gcttgcggcaatcgtgtctaa

SEQ ID NO: 12 - DNA, 22 bp, Artificial Sequence
  BMPRI reverse primer
gcagcctgtgaagatgtagagg

SEQ ID NO: 13 - DNA, 24 bp, Artificial Sequence
  BMPRII forward primer
cacaccagccttatactctagata

SEQ ID NO: 14 - DNA, 24 bp, Artificial Sequence
  BMPRII reverse primer
cacatatctgttatgaaacttgag